(19) United States
(12) Patent Application Publication
(10) Pub. No.: US 2008/0101543 A1
(43) Pub. Date: May 1, 2008

(54) X-RAY DIAGNOSTIC APPARATUS USING IMAGE SENSOR

(75) Inventors: Ki Bong Sung, Gyeonggi-Do (KR); Jong Lae Park, Seoul (KR)

Correspondence Address: LOWE HAUPTMAN HAM & BERNER, LLP, 1700 DIAGONAL ROAD, SUITE 300, ALEXANDRIA, VA, 22314, US

(73) Assignee: POSKOM CO., LTD., GYEONGGI-DO (KR)

(21) Appl. No.: 11/678,483

(22) Filed: Feb. 23, 2007

(30) Foreign Application Priority Data
Oct. 30, 2006 (KR) 10-2006-0105839

(51) Int. Cl. G21K 1/02; A61B 6/00

(52) U.S. Cl. 378/147; 378/4

(57) ABSTRACT

The present invention relates to an X-ray diagnostic apparatus, and more particularly, to an X-ray diagnostic apparatus that can confirm an area on which X-ray is illuminated, using an image sensor, prior to photographing the X-ray of a wounded portion of a patient or an animal. The X-ray diagnostic apparatus according to the present invention can confirm the area on which the X-ray is illuminated, through the image sensor, thereby decreasing the amount of heat generated and saving the power consumption required to operate the lamp, compared with the conventional X-ray diagnostic apparatus. Also, the X-ray diagnostic apparatus according to the present invention can combine, edit and provide the real image photographed by the X-ray, the image to be photographed through the X-ray, or the image of a patient to exactly and conveniently diagnose the health state of the patient.

### CROSS-REFERENCE TO RELATED PATENT APPLICATION

This application claims the benefit of Korean Patent Application No. 10-2006-105839, filed on Oct. 30, 2006, in the Korean Intellectual Property Office, the disclosure of which is incorporated herein in its entirety by reference.

### BACKGROUND OF THE INVENTION

1. Field of the Invention

The present invention relates to an X-ray diagnostic apparatus, and more particularly, to an X-ray diagnostic apparatus that can confirm an area on which X-ray is illuminated, using an image sensor, prior to photographing the X-ray of a wounded portion of a patient or an animal.

2. Description of the Related Art

An X-ray diagnostic apparatus is used for diagnosing a health state of a patient or an animal by illuminating X-ray to the patient or the animal. The conventional X-ray diagnostic apparatus includes an X-ray tube for generating X-ray, a collimator for controlling an area on which the X-ray is illuminated, and a film for developing the X-ray that has passed through an inner body of the patient or the animal. Before illuminating the generated X-ray to the body of the patient or the animal, the area on which the X-ray is illuminated should be checked in order to exactly photograph the wound portion of the patient or the animal using the collimator.

FIG. 1 schematically shows an X-ray collimator according to the related art.

In order to limit an illumination area of the X-ray generated from an X-ray tube 1, shutter 3 is symmetrically disposed above and below an illumination axis 6 of the X-ray. In general, the shutter for limiting the length of an area on which X-ray is illuminated, and the shutter for limiting the width of an area on which X-ray is illuminated are symmetrically disposed above and below and at the left and right of the illumination axis 6 of the X-ray. For the convenience of description, FIG. 1 shows only the shutter 3 symmetrically disposed above and below the illumination axis 6 of the X-ray. The area A on which the X-ray is illuminated is increased or decreased by increasing or decreasing a symmetric distance D between the illumination axis 6 and the shutter 3.

Meanwhile, the X-ray collimator is provided with a reflection mirror 2 disposed diagonally with respect to the illumination axis 6 of the X-ray and a lamp 5 facing a reflection surface of the reflection mirror 2 so as to estimate an illuminated area where the X-ray will be illuminated. The reflection mirror 2 reflects a light of the lamp 5 to a direction of the illumination axis 6 of the X-ray. The reflected light of the lamp 5 passes through the shutter 3 and illuminates the same area as the area A where the X-ray is illuminated. Accordingly, it is possible to confirm the illuminated area A of the X-ray using the light of lamp 5, prior to photographing the X-ray of the patient or the animal.

The aforementioned conventional X-ray collimator uses the light of the lamp 5 to estimate the illuminated area A. Thus it is difficult to exactly discriminate the illuminated area A in a bright indoor space or the fields and accordingly it is possible to confirm the illuminated area A only in a dark indoor space. To overcome the above drawback, a high luminance lamp is used, so that much heat is generated from an inside of the collimator and much power is consumed.

### SUMMARY OF THE INVENTION

The present invention provides an X-ray collimator incorporating an image sensor.

The present invention also provides an X-ray diagnostic apparatus that can confirm an illumination area of an X-ray using a collimator provided with an image sensor.

According to an aspect of the present invention, there is provided a collimator including: shutters symmetrically disposed in the center of an illumination axis of X-ray to limit area on which the X-ray is illuminated; an image sensor sensing a light inputted from the area on which the X-ray is illuminated, and photographing an image of the area on which the X-ray is being illuminated; and a reflection mirror diagonally disposed with respect to the illumination axis of the X-ray such that the light inputted into the shutters is reflected toward the image sensor.

According to another aspect of the present invention, there is provided an X-ray diagnostic apparatus including: an X-ray collimator photographing an image of an area on which X-ray is illuminated, using an image sensor; and an image output unit converting the image photographed by the image sensor into a predetermined image format and displaying the format-converted image.

Preferably, the image output unit according to one embodiment of the present invention includes: an image processing unit converting the image photographed by the image sensor into a predetermined image format; a display unit displaying the format-converted image; and a storage unit storing a data of the format-converted image.

### BRIEF DESCRIPTION OF THE DRAWINGS

The above and other features and advantages of the present invention will become more apparent by describing in detail exemplary embodiments thereof with reference to the attached drawings in which:

FIG. 1 schematically shows an X-ray collimator according to the related art;

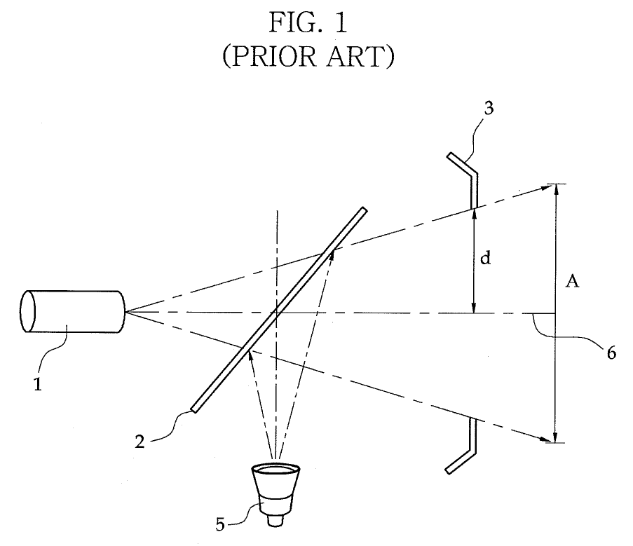

FIG. 2 shows an X-ray collimator according to the present invention; and

FIG. 3 shows a functional block diagram of an X-ray diagnostic apparatus according to one embodiment of the present invention.

### DETAILED DESCRIPTION OF THE INVENTION

The present invention will now be described more fully with reference to the accompanying drawings, in which exemplary embodiments of the invention are shown.

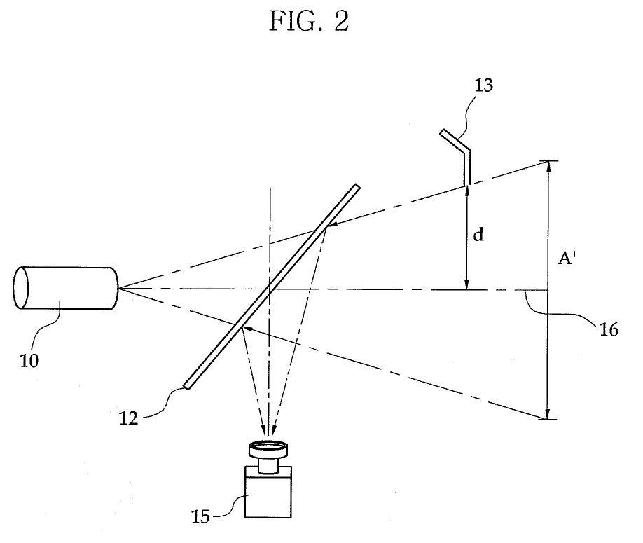

FIG. 2 shows an X-ray collimator according to the present invention.

Referring to FIG. 2, an X-ray collimator is provided in a housing (not shown) thereof with a reflection mirror 12, shutter 13 and an image sensor 15. The shutter 13 is symmetrically disposed in center of an illumination axis 16 of an X-ray tube 10. In order to limit an area A′ on which the X-ray is illuminated, the shutter for limiting a length of the area A′ on which X-ray is illuminated, and the shutter for limiting a width of the area A′ on which X-ray is illuminated are symmetrically disposed above and below and at the left and right of the illumination axis 16 of the X-ray. For the convenience of description, FIG. 2 shows only the shutter 13 symmetrically disposed above and below the illumination axis 16 of the X-ray.

An image of the area A′ on which X-ray will be illuminated is positioned outside the housing and a light is inputted from the area A′ into the reflection mirror 12 through the shutter 13.

The image sensor 15 is disposed facing a reflection surface of the reflection mirror 12 and the light inputted into the reflection mirror 12 is reflected toward the image sensor 15. The reflection mirror 12 is diagonally disposed with respect to the illumination axis 16 of the X-ray so as to reflect the light inputted from the area A′ toward the image sensor 15. Preferably, the reflection mirror 12 is diagonally disposed at 45 degree with respect to the illumination axis 16 of the X-ray.

The image sensor 15 is a sensor for sensing an image information, and is representatively implemented by a charge-coupled device (CCD) and a complementary metal oxide semiconductor (CMOS). The image sensor 15 includes a lattice type photosensitive emulsion for converting light into charge, and measures how much light is exposed to the respective photosensitive emulsion. Photons accumulated in the photosensitive emulsion are converted into electrons and the electrons are again converted into a charge.

The image of the area A′ where the X-ray will be illuminated is transmitted to the reflection mirror 12 in the form of light, and the image of the area A′ transmitted to the reflection mirror 12 in the form of light is reflected toward the image sensor 15. The image sensor 15 converts the reflected light into a charge and obtains the image data of the area A′ from the converted level of charge.

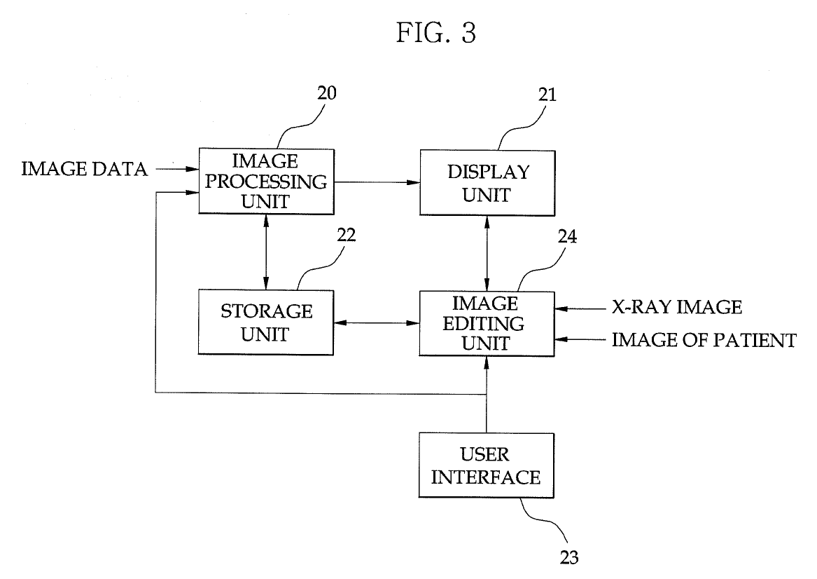

FIG. 3 shows a functional block diagram of an X-ray diagnostic apparatus according to one embodiment of the present invention.

Referring to FIG. 3, the obtained image data through the image sensor 15 are inputted into an image processing unit 20. The image processing unit 20 filters the image data with low-frequency and converts the obtained image data into an image data having a predetermined format corresponding to a display unit 21 and transmits the same to the display unit 21.

The display unit 21 receives the converted image data from the image processing unit 20 to display the image of the area A′ such that a user can confirm the area A′ where the X-ray will be really illuminated in a body of a patient or animal through the displayed image, prior to photographing the X-ray of the patient or the animal. The image displayed on the display unit 21 is stored in a storage unit 22 according to a user command inputted through a user interface 23.

Meanwhile, an image editing unit 24 combines and edits an image photographed by the X-ray and the displayed image or combines and edits an image of a patient or animal photographed in advance through the image sensor 15 and the image photographed by the X-ray or the displayed image according to the user command inputted through the user interface 23. The image edited by the image editing unit 24 is stored in the storage unit 22 according to the user command inputted through the user interface 23.

As described above, the X-ray diagnostic apparatus according to the present invention can in advance confirm the area A′ on which the X-ray will be illuminated, through the image sensor, thereby decreasing the amount of heat generated and saving the power consumption required to operate the lamp, compared with the conventional X-ray diagnostic apparatus.

Also, the X-ray diagnostic apparatus according to the present invention can combine, edit and provide the image photographed by the X-ray, the image to be photographed through the X-ray, or the image of a patient to exactly and conveniently diagnose the health state of the patient or the animal.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. An X-ray diagnostic apparatus comprising:
an X-ray collimator photographing an image of an area on which X-ray is illuminated, using an image sensor; and
an image output unit converting the image photographed by the image sensor into a predetermined image format and displaying the format-converted image.

2. The X-ray diagnostic apparatus of claim 1, wherein the X-ray collimator comprises:
shutters symmetrically disposed in center of an illumination axis of the X-ray to limit the illumination area of the X-ray;
the image sensor photographing the image of the area on which the X-ray is illuminated, through a light inputted into the shutters from the limited illumination area of the X-ray; and
a reflection mirror diagonally disposed with respect to the illumination axis of the X-ray such that the light inputted into the shutters is reflected toward the image sensor.

3. The X-ray diagnostic apparatus of claim 2, wherein the image output unit comprises:
an image processing unit converting the image photographed by the image sensor into a predetermined image format;
a display unit displaying the format-converted image; and
a storage unit storing a data of the format-converted image.

4. The X-ray diagnostic apparatus of claim 3, wherein the image output unit further comprises an image editing unit editing an image photographed by the X-ray and an image photographed by the image sensor.

5. An X-ray collimator comprising:
shutters symmetrically disposed in center of an illumination axis of X-ray to limit an illumination area of the X-ray;
an image sensor photographing the image of the area on which the X-ray is illuminated, through a light inputted into the shutters from the limited illumination area of the X-ray; and
a reflection mirror diagonally disposed with respect to the illumination axis of the X-ray such that the light inputted into the shutters is reflected toward the image sensor.
